SEQUENCE LISTING (5 sequences)

SEQ ID NO: 1 - DNA, 2024 bp, Rats
  CDS (146)..(1243)
gaccacggaaggttgaaggataatcttctagtaccagcactttgaatgagaatttgtttctctgccacaaactgatttttttttttaaattttatttttctggtggaggagttgaaacaaacctaccttttgtggcatcgcagctatgcagcttgaaattcaagtagcactaaattttattatttcctatttgtacaataagcttcccaggagacgtgtcaacatttttggtgaagaacttgaaagacttcttaaacagaaatatgaagggcattggtatcctgagaagccatacaaaggctcagggtttagatgcatacacgtaggggagaaggtggacccggtgatcgagcaagcgtccaaagagagtggtctggacattgacgatgttcgtggcaatctgccgcaggatctgagcgtgtggatcgacccgtttgaggtttcctaccaaatcggtgagaagggaccagtgaaggtgctgtacgtagatgacagtaatgagaacgggtgtgagctggataaggagatcaagaacagctttaaccctgaggcccaggttttcatgcccataagcgacccagcctcctctgtgtccagctcgccgtcgcctccctttggccattctgctgccgtcagccccaccttcatgccccggtccactcagcctttaacctttaccactgccacttttgctgccaccaagtttggctccaccaaaatgaagaacagtggccgtagcagcaaggtagcacgcacttctcccatcagcctgggcctgaatgtcaatgtgaacgacctcctgaagcagaaagccatctcttcctcaatgcactctctgtacgggctgggcctgggcagccagcagcagcctcagccgcagccgcagcagccgccatcccagccaccaccgccaccaccacctccacagcagcagcagcagcatcagcagcagcagcagcagcagcaacagcagcagcagcagccgcagcagcaaacctctgctctttctcccaatgccaaggaatttatttttcctaacatgcagggtcaaggtagtagtaccaatggaatgttcccaggtgacagcccccttaacctcagtcccctccagtacagtaatgcctttaatgtgtttgcggcctacggaggcctcaacgagaagtctttcgtagacggcttgaattttagcttaaataacattcagtattctaaccagcagttccagcccgtaatggctaactaaaaaaacaggaaaagagagaacatgtgtcgtacaagttaaaatgcatcggcccaagggggacttttttttttttttgcctccttgagattttttttttaagcttatagtaaggatacattcaagcttggtcacaaaataaaacatgcatcttttttcatttgccaaccaagcacaatgttattttatactgactgtatattttaaagtatactttcagatatggcctcttacagtatttaagatatagcaaggacatggctgatttttttttatatatatatataaaaaattggcactaataagtgggtttattggtcttttctaattgtataatttaatttagtacaaagtttgtaaactatcagaggatatatatatatatacatatatatattgtttctacgacacggtattgcatttctatctttttactacagtgatctgtggcagcggcttcatgttgtattttttttactgaaattgtaaaatatccatcttaaagacatcaactattctgaaaattgtgtacaggatattcctttagtggtggaattaaaatgtacgaatatttgctttttcaaaaaaatgtattttctgttaaaggtttaaagatttttgctatatattatggaagaaaatgtaatcgtaaatattaattttgtacctatattgtgcaatacttgaaaaaaaaacggtataaaagtattttgagtcagtgtcttacatgttaagagggactgaaatagtttatattaagtttgtattaaaattctttaaaatt

SEQ ID NO: 2 - protein, 365 aa, Rats
MQLEIQVALNFIISYLYNKLPRRRVNIFGEELERLLKQKYEGHWYPEKPYKGSGFRCIHVGEKVDPVIEQASKESGLDIDDVRGNLPQDLSVWIDPFEVSYQIGEKGPVKVLYVDDSNENGCELDKEIKNSFNPEAQVFMPISDPASSVSSSPSPPFGHSAAVSPTFMPRSTQPLTFTTATFAATKFGSTKMKNSGRSSKVARTSPISLGLNVNVNDLLKQKAISSSMHSLYGLGLGSQQQPQPQPQQPPSQPPPPPPPPQQQQQHQQQQQQQQQQQQQPQQQTSALSPNAKEFIFPNMQGQGSSTNGMFPGDSPLNLSPLQYSNAFNVFAAYGGLNEKSFVDGLNFSLNNIQYSNQQFQPVMAN

SEQ ID NO: 3 - DNA, 20 bp, Rats
acttggatttcaagctgcat

SEQ ID NO: 4 - DNA, 20 bp, Rats
acttctcgttgaggcctccg

SEQ ID NO: 5 - DNA, 20 bp, Rats
gactgacatgcgattgagct

(19) United States
(12) Patent Application Publication
(10) Pub. No.: US 2004/0242462 A1
(43) Pub. Date: Dec. 2, 2004

(54) Function and application of tob gene in central nervous systems of mammal

(76) Inventors: Jing Naihe, Shanghai (CN); Mei Lei Jin, Shanghai (CN)

Correspondence Address: Jules E Goldberg, Reed Smith, 599 Park Avenue, New York, NY, 10022, US

(21) Appl. No.: 10/469,483

(22) PCT Filed: Feb. 26, 2002
(86) PCT No.: PCT/IB02/02042

(30) Foreign Application Priority Data
Feb. 27, 2001 (CN) 01-1-05448.4

(51) Int. Cl. C12Q 1/68; G01N 33/567; A61K 38/40

(52) U.S. Cl. 514/6; 435/6; 435/7.21

(57) ABSTRACT

This invention concerns the mammalian Tob gene and its coding products, as well as its uses in such areas as the diagnosis and treatment of hypomnesia and amnesia. In addition, this invention also concerns pharmaceutical compositions and tonics containing the Tob protein, as well as the uses of Tob in the screening of drugs concerned with improving memory and treating amnesia or raising movement coordination.

This invention concerns the areas of bio-engineering and medicine. In concrete terms, this invention concerns the mammalian Tob gene and its coding products, as well as its uses in such areas as the diagnosis and treatment of hypomnesia and amnesia. In addition, this invention also concerns pharmaceutical compositions and tonics containing the Tob protein, as well as the uses of Tob in the screening of drugs concerned with improving memory and treating amnesia or raising movement coordination.

It is already known that the ErbB-2 gene codes for a receptor-type protein tyrosinekinase (RPTK), which plays an important role in cell growth and division. Its excessive expression can lead to the occurrence of tumours, but its mechanism in this still awaits elucidation.

In 1996, Japanese researchers discovered a new protein molecule which interacts with the ErbB-2 gene product p185erbB2-Tob (Transducer of ErbB-2), and cloned a human Tob gene cDNA sequence. Tob has a definite homology with the already known anti-replication gene BTG-1. Research has shown that similarly to BTG-1, Tob also manifests an biting action on cell growth, but its action is countered by p185erbB2. Other members of the Tob/BTG-1 family were subsequently discovered one after another in humans and mice, but research into their functions was limited to the inhibition of cell replication.

Learning and memory are basic functions of the brain. In the higher animals, the hippocampi are medial temporal lobe brain structures, and after their destruction, new memories cannot be formed. The synapses of the hippocampi have very strong plasticity and can be induced to produce and maintain long-term potentiation (LTP), and if any kind of protein coding gene linked to LTP induction or maintenance is knocked out, there will be no further LTP phenomenon in the hippocampi, and the animal will not be able to form memories. The hippocampi are therefore important structures in learning and memory, and LTP acts as a synaptic mechanism for learning and memory, something which has already been widely accepted by neurologists.

However, up to now there are very few proteins which are known to participate in and affect memory. It is therefore vital in this area to discover new proteins related to memory and to develop and improve drugs which can improve memory or treat amnesia.

The objective of this invention is therefore to provide a protein related to memory—Tob protein, and its uses in diagnosing, improving or treating hypomnesia and amnesia.

Another objective of this invention is to provide pharmaceutical compositions and tonics containing the Tob protein.

In the first aspect of this invention, a method has been provided for diagnosing individual susceptibility to amnesia, which includes the steps:

Detecting the individual's Tob gene, transcript and/or protein and comparing with the normal Tob gene, transcript and/or protein.

Differences present indicate that the probability of that individual suffering from amnesia is higher than that of the normal population.

Preferably that which is detected is the human Tob gene or transcript, and it is compared to a normal human Tob nucleotide sequence for any difference.

In the second aspect of this invention, a method for treating amnesia is provided, which includes the step of using a safe and effective dose of normal Tob protein on the patient requiring the said treatment.

In the third aspect of this invention, a method for improving the memory or movement coordination of mammals is provided, which includes the step of using a safe and effective dose of Tob protein on the subject requiring it.

In the fourth aspect of this invention, tonics are provided which include a mammalian Tob protein or active fragments thereof.

In the fifth aspect of this invention, pharmaceutical compositions are provided which include a safe and effective dose of mammalian Tob protein and a pharmaceutically acceptable carrier. Preferably, the said Tob protein is selected from the following groups of animal Tob protein: human, rat and mouse.

In the sixth aspect of this invention, a reagent kit for detecting susceptibility to amnesia is provided, which includes an initiator for the specific amplification of the Tob gene or transcript, or an antibody specifically binding with the Tob protein.

In the seventh aspect of this invention, a method for screening drugs for the treatment of amnesia is provided, which includes the steps:

(1) Inserting the Tob gene cDNA into an expression vector, transfecting a mammalian cell strain and preparing a cell strain to express the Tob protein;

(2) Adding a test compound to the culture fluid for the cell strain expressing the Tob protein in step (1) to detect changes in the amount of Tob protein expressed; the compound promoting the increase in the amount of Tob protein expression being the drug awaiting screening for the treatment of amnesia.

In the eighth aspect of this invention, a type of isolated Tob protein is provided which includes the amino acid sequence indicated by SEQ ID NO: 2.

In the appended figures,

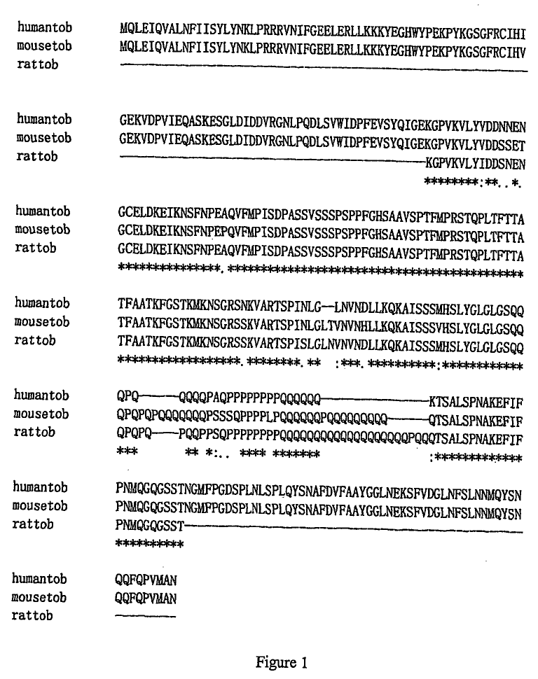

FIG. 1 shows a comparison of the human, mouse and rat Tob protein conservative regions. In the figure, humantob is human Tob; mousetob is mouse Tob; and rattob is rat Tob.

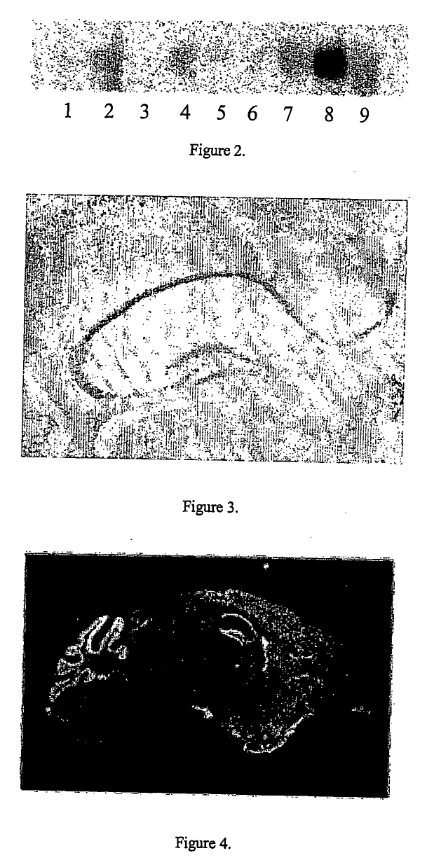

FIG. 2 is an expression spectrum of rat Tob mRNA. Lanes 1-9 are: brain, hippocampus, heart, intestines, kidneys, liver, lungs, muscle and spleen respectively.

FIG. 3 is a distribution diagram of rat Tob mRNA in the hippocampus. Tob mRNA is mainly detected in the DG, CA1 and CA3 regions of the hippocampus.

FIG. 4 is a distribution diagram of mouse Tob mRNA in the brain. Tob mRNA is mainly detected in the hippocampus and cortex of the cerebellum.

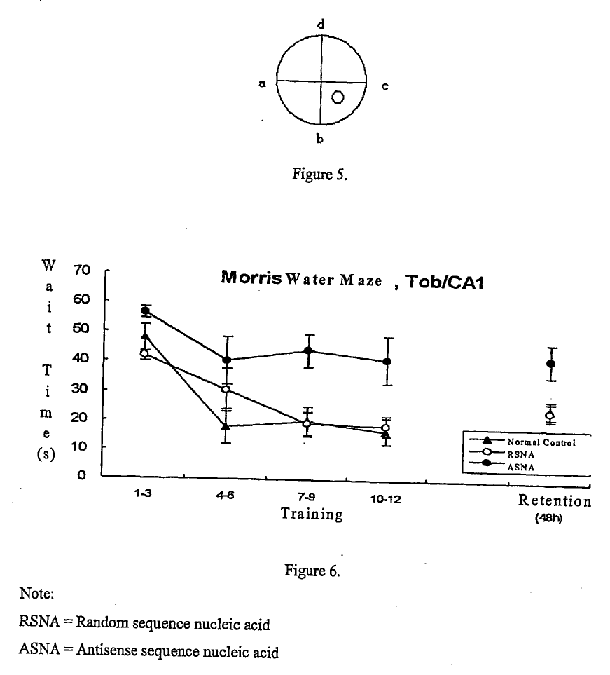

FIG. 5 is a schematic diagram of the water maze, in which “O” indicates the location of the concealed platform.

FIG. 6 shows the effect of Tob gene antisense sequence nucleic acid on learning and memory maintenance in the Morris water maze by the rat.

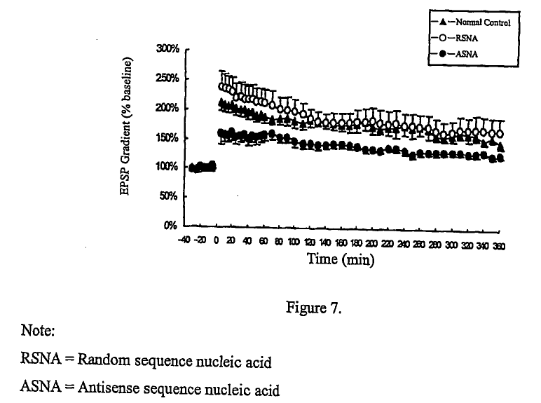

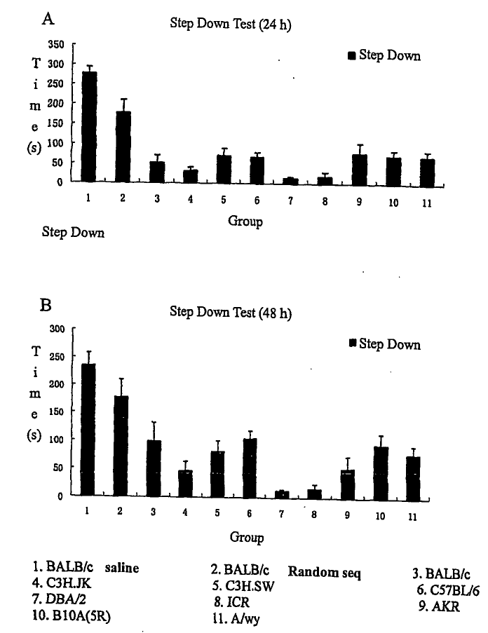

FIG. 7 shows the LTP recorded in the CA1 region of the hippocampus of the rats in the control group and the antisense nucleic acid groups.

FIG. 8 shows the negative effect in all cases on the memory of nine different strains of mice by the Tob antisense nucleic acid sequence. Group 1 is the BALB/c mouse+saline; Group 2 is BALB/c mouse+Tob random sequence; and Groups 3-9 are different strains of mice+Tob antisense sequence.

As used in this document, the technical terms “Tob protein” and “Tob polypeptide” can be used interchangeably to indicate polypeptides which are specifically expressed in the mammalian hippocampus, the amino acid sequences of which have over 80% homology with human, rat or mouse Tob amino acid sequences, and preferably over 85% and better still over 90%. Also, Tob active fragments and conservative polypeptides or active derivatives can be used for this invention.

After broad and deep research, the present inventor has not only cloned rat Tob cDNA sequences but has also used different animal behaviour models to study the Tob gene function in learning and memory processes. It was accidentally discovered that Tob is connected with memory and movement coordination.

A pair of initiators was designed with reference to human and mouse Tob 1 gene cDNA conservative regions, and a section of DNA sequence was cloned through PCR in an 8 week-old rat cDNA library. The sequencing results showed that the length of this sequence was 604 bp and that it had 91% and 90% homology with the corresponding regions of human and mouse Tob 1 cDNA respectively (FIG. 1). With that sequence section as a probe, the entire length of the rat Tob gene cDNA was cloned through screening the adult rat brain cDNA library. The results of Northern blotting hybridisation showed that the rat Tob gene expression mode was similar to that of humans and mice (FIG. 2). The results of in situ hybridisation showed that the Tob gene has a specific expression in the neurons of the hippocampus in rats (FIG. 3).

With rats as the experimental animals, a functional measurement was made of the Tob gene through such behavioural experimental models as the water maze and environmental fear (Fear-conditioning to Context). The Tob gene antisense nucleic acid was injected into a fixed position in the CA1 region of the hippocampus, from which it was discovered that the rat Tob gene antisense nucleic acid can significantly inhibit learning and memory in the rat (FIG. 6); and it inhibited the long-term potentiation of synapses in the CA1 region (LTP; FIG. 7).

With mice as the experimental animals, mouse Tob gene antisense nucleic acid was regularly injected into the lateral ventricles of the mouse using a Hamilton microsyringe with a fixed dose, and control groups were formed which were injected with physiological saline and randomly sequenced nucleic acid (Scramble). Then the experimental group and the control groups were examined in the step-down test, and statistical analysis was carried out on the data obtained. The results showed that the memory of the experimental group (injected with Tob antisense nucleic acid) suffered a significant reduction in comparison with the control groups. In order further to confirm the connection between the Tob gene and learning and memory, 9 pairs of experimental mice of different strains were selected for functional rescreening. The results showed that the Tob gene antisense nucleic acid could significantly inhibit the mouse learning and memory for all the strains (FIG. 8).

In addition, in view of the specific expression of the Tob mRNA in the cortex of the cerebellum, it is suggested that the Tob protein is connected with movement coordination (FIG. 4).

Based on a comparison of the protein homology, human Tob protein has a high level of homology with mouse and rat Tob protein (they are over 90% identical). The structure of mammalian Tob protein and the specificity of tissue distribution indicate that Tob protein has the same functions in different mammals, namely to improve memory and raise movement coordination. Therefore human Tob protein is similarly connected with human memory and movement coordination, and drugs and diagnosis and treatment techniques designed on the basis of the human Tob gene and its expression products can be used for the diagnosis and treatment of human amnesia and can improve memory and raise movement coordination.

The entire sequence length of Tob nucleotides or fragments thereof can usually be obtained by the methods of PCR amplification, recombination or artificial synthesis. As regards the PCR amplification method, the initiator can be designed on the basis of the already known human Tob, mouse Tob or the rat Tob nucleotide sequences published in this invention and particularly the Open Reading Frame sequence. Also commercially available cDNA libraries or a cDNA library prepared by the conventional methods already known to technicians in this field can also be used as a template to amplify and obtain the relevant sequence. When the sequence is relatively long, it is usually necessary to carry out two or more PCR applications and then connect the fragments obtained by the different amplifications and accurately join them together in sequence.

As soon as the relevant sequence has been obtained, it is possible to use the recombination method to obtain the relevant sequence in large quantities. This is usually through cloning it into a vector, transferring it to a cell and then using the conventional method of separating out the relevant sequence from the replicated host cells.

It is also possible to use artificial synthesis to synthesise the relevant sequence, particularly if the fragment length is relatively short. It is usually done by first synthesising many small fragments and then joining them together to obtain fragments containing very long sequences.

It is currently possible to obtain coded protein DNA sequences of this invention (or their fragments or derivatives) completely through chemical synthesis. It is then possible to introduce these DNA sequences into all the various existing DNA molecules known in this field (or vectors) and into cells. Furthermore, it is also possible through chemical synthesis to introduce mutations into the protein sequence of this invention.

Tob protein usable in this invention can be obtained by inserting the corresponding code sequence into a host cell (direct introduction or through the introduction of a vector containing the Tob code sequence) and culturing the transfected host cell under appropriate conditions to express the Tob protein and then isolating and purifying the Tob protein.

In concrete terms, the Tob protein or polypeptide of this invention has many uses. These uses include (but are not limited to): direct use as drugs for the treatment of diseases caused by a drop in or loss of the Tob protein function (such as hypomnesia), and their use for screening antibodies, polypeptides or other ligands which promote the Tob protein function. The use of expressed recombinant Tob protein for screening polypeptide libraries can be applied to the search for polypeptide molecules having treatment value in stimulating the human Tob protein function.

On another matter, this invention also includes polyclonal antibodies and monoclonal antibodies having specificity for the Tob DNA or polypeptides coded by fragments thereof, and particularly monoclonal antibodies. Here, “specificity” indicates that the antibody can bind with the Tob gene product or fragment. It preferably indicates those antibodies which can bind with the Tob gene product or fragment but do not identify and bind with other unrelated antigen molecules. Antibodies in this invention include those molecules which can bind with and inhibit Tob protein and also include those antibodies which do not affect the Tob protein function.

This invention not only includes complete monoclonal or polyclonal antibodies but also includes antibody fragments having immune activity, such as Fab′ or (Fab)₂ fragments; antibody heavy chains; antibody light chains; or embedded antibodies, such as antibodies which have rodent antibody binding specificity but still retain the antibody parts from humans.

The antibodies of this invention can be prepared through all sorts of techniques already known to technicians in this field. For example, purified Tob gene products or fragments having antigenicity can be used on animals for inducing the production of polyclonal antibodies. Similarly, cells expressing Tob protein or fragments thereof having antigenicity can be used for immunising animals to produce antibodies. Monoclonal antibodies of this invention can be prepared using hybridoma techniques (see Kohler et al, Nature 256; 495, 1975; Kohler et al, Eur. J. Immunol. 6: 511, 1976; Kohler et al, Eur. J. Immunol. 6: 292, 1976; Hammerling et al, In Monoclonal Antibodies and T Cell Hybridomas, Elsevier, N.Y., 1981). The various antibodies of this invention can be obtained using for example human, rat or mouse Tob gene product fragments or functional regions through conventional immunotechnology. These fragments or functional regions can be prepared using the recombinattion method or can be synthesised using polypeptide synthesis instruments. Antibodies which bind with unmodified Tob gene product forms can be produced by immunising animals with gene products produced in prokaryocytes (such as E. coli); antibodies binding with translated modified forms (such as saccharified or phosphated proteins or polypeptides) can be obtained by immmunising animals with gene products produced in eukaryocytes (such as yeast or insect cells).

Anti-Tob protein antibodies can be used in immuno-histochemical techniques to detect Tob protein in live test specimens.

Production of polyclonal antibodies can be done using animals immune to Tob protein or polypeptides, such as guinea-pigs and sheep. Many adjuvants can be used to strengthen the immune response, including but not limited to Freund's adjuvant.

By utilising the protein of this invention in all kinds of conventional screening methods, it is possible to screen out substances which interact with Tob protein, such as inhibitors, exciters or antagonists. At the time of screening, the Tob protein can be included in the biological analysis and measurement, and by measuring the interaction between the Tob protein affected by the compound and its receptor, it can be determined whether the compound is an antagonist. It is furthermore also possible to use the test compound and the Tob protein together on experimental animals and to compare with the control group for the presence of changes in the animals' memory and thus to determine whether the compound is a Tob protein exciter or antagonist.

In addition, it is also possible to insert the Tob gene cDNA into an expression vector and transfect a mammalian cell strain to prepare a cell strain with highly expressed Tob protein; and with the Tob protein in this cell as the target location, it is possible to screen for drugs which have an exciting or inhibiting action on the Tob protein. Also a test compound can be added to the culture fluid for the cell strain expressing the said Tob protein to detect changes in the Tob protein expression level. Compounds which promote Tob protein expression are drugs available for selection for the treatment of amnesia, and compounds which inhibit the promotion of Tob protein expression can be used as drugs to help people forget unwelcome recollections.

The protein of this invention and its antibodies, inhibitors, exciters and antagonists can provide different effects when used for treatment (drugs). Usually, these substances can be made up with non-toxic, inert and pharmaceutically acceptable aqueous carrier media in which the pH is usually approximately 5-8, and ideally approximately 6-8, and indeed the pH value can be varied according to the nature of the substances being added and the disease awaiting treatment. A properly constituted pharmaceutical composition can be administered via the conventional routes including (but not limited to): intramuscular, intravenous, subcutaneous, oral or topical.

Normal Tob polypeptides can be used directly for the treatment of disease, for example in the treatment of amnesia When using the Tob protein of this invention, it is also possible to use other drugs for the treatment of amnesia at the same time.

This invention also provides pharmaceutical compositions, which include a safe and effective dose of the Tob protein of this invention as well as a pharmaceutically acceptable carrier or excipient. This kind of carrier includes (but is not limited to): saline solution, buffer solution, glucose, water, glycerine, ethanol, and mixtures of the same. A pharmaceutical preparation should match its method of administration. The pharmaceutical compositions of this invention can be made up as injectables, for example being prepared by conventional methods using physiological saline or an aqueous solution containing glucose and other supplementary materials. Tablet or capsule forms of the pharmaceutical compositions can be prepared by conventional methods. Pharmaceutical compositions, whether as injectables, solutions, tablets or capsules, should be prepared under sterile conditions. The dose of the active ingredient is the effective dose for treatment, for example approximately 0.1 μg/kg of bodyweight per day—approximately 5 mg/kg of bodyweight In addition, the polypeptides of this invention can also be used together with other treatments.

When using a pharmaceutical composition, a safe and effective dose of the Tob protein or its antagonist or exciter is used on mammals, in which the safe and effective dose is usually at least approximately 0.1 μg/kg of bodyweight. In addition, under most circumstances, approximately 10 mg/kg of bodyweight should not be exceeded, and the dose should preferably be approximately 0.1 μg/kg of bodyweight—approximately 100 μg/kg of bodyweight ¹. Of course, such factors as the route of administration and the patient's health should also be considered when determining the actual dose. These are all within the skill range of experienced doctors. ¹ Translator's note: there appears to be something amiss with these numbers, but the units have been correctly translated.

Tob protein polynucleotides can also be used for many treatment objectives. Genetic treatment techniques can be used to treat abnormalities in cell replication, development or metabolism caused by non-expression of Tob protein or abnormal/inactive Tob protein expression. The method for constructing a Tob gene recombinant viral vector can be seen in the existing literature (Sambrook et al). Also recombinant human Tob genes can be packaged in liposomes and then transferred to cells.

The method of inserting polynucleotides into tissue or cells includes: directly injecting the polynucleotide into body tissue; or first introducing the polynucleotide into the cell in vitro by means of a vector (such as a virus, bacteriophage or plasmid) and then transferring the cell into the body.

This invention also concerns diagnosis and test methods involving the quantitative and locational measurement of Tob protein levels. These tests are familiar in this field and include FISH measurement and radioimmunoassay. The measured Tob protein levels in these tests may be used to explain the importance of the Tob protein in various illnesses and for the diagnosis of illnesses in which the Tob protein plays a part.

One method for detecting whether the Tob protein is present in a specimen is to detect the specific antibody for the Tob protein and includes: bringing the specimen into contact with the Tob protein specific antibody; and observing whether an antibody complex is formed, the formation of such an antibody complex indicating that the Tob protein is present in the specimen.

The Tob protein polynucleotide can be used in the diagnosis and treatment of diseases connected with the Tob protein. In diagnosis, the Tob protein polynucleotide can be used to detect the Tob protein expression or the abnormal expression of the Tob protein during illness. Thus, the Tob DNA sequence can be used in the hybridisation of live test specimens to determine whether the Tob protein expression is abnormal. The hybridisation technology includes the Southern blotting method, the Northern blotting method and in situ hybridisation. These technological methods are published and mature technologies, and the corresponding reagent kits can all be obtained through commercial channels. The polynucleotide of this invention can partialaly or completely serve as a probe fixed in a microarray or DNA core (also referred to as a “gene core”). They can be used to analyse anomalous genetic expression in tissue and in genetic diagnosis. Tob gene transcription products can also be detected by in vitro amplification using an initiator specific to Tob to carry out an RNA-polymerase chain reaction (RT-PCR).

The detection of Tob gene mutations can also be used in the diagnosis of diseases related to Tob protein. The forms of Tob protein mutations include point mutations, dislocations, deletions, recombinations and any other abnormalities in comparison with normal wild-type Tob DNA sequences. Existing techniques can be used for detection of mutations, such as Southern blotting, DNA sequencing, PCR and in situ hybridisation. Also, mutations may affect protein expression, and so using the Northern blotting method and Western blotting method may indirectly determine whether the gene shows any mutations.

The Tob protein of this invention can not only be used in the treatment of hypomnesia or handicapped subjects, but can also be used to improve the memory of normal individuals. This invention therefore also provides tonics which include mammalian Tob protein or its active fragments. The tonics of this invention can be prepared by conventional tonic preparation methods by mixing mammalian Tob protein or its active fragments with a suitable diluent or food etc. The preferred forms for the tonic are as tablets, granules and oral preparations.

This invention is further described below in combination with actual practical examples. It should be understood that these practical examples are only used to explain this invention and may not be used to limit the scope of this invention. Experimental methods under actual conditions are not indicated in the following practical examples, and they will usually be carried out under conventional conditions such as those related in Sambrook et al, Molecular Cloning: Laboratory Handbook (New York: Cold Spring Harbor Laboratory Press, 1989) or according to the conditions recommended by the manufacturer.

### PRACTICAL EXAMPLES

### Practical Example 1

Obtaining Rat Tob cDNA Sequence

An initiator was designed through PCR with human and mouse Tob gene cDNA conservative region as the reference, and a section of DNA sequence was cloned in an 8 week-old rat brain cDNA library (constructed by conventional methods). With this section of the sequence as a probe, rat Tob gene whole length cDNA was obtained through screening the 8 week-old rat brain cDNA library. The sequencing results showed that this cDNA length was 2024 bp, and the actual sequence was as shown in SEQ ID NO: 1. In this the Open Reading Frame (ORF) was 146˜1243 bp, and the coded rat Tob protein contained 365 aa, the sequence being as follows:

| | | | | | | | |
|---|---|---|---|---|---|---|---|
| MQLEIQVALN | FIISYLYNKL | PRRRVNIFGE | ELERLLKQKY | EGHWYPEKPY | KGSGFRCIHV | 60 | (SEQ ID NO: 2) |
| GEKVDPVIEQ | ASKESGLDID | DVRGNLPQDL | SVWIDPFEVS | YQIGEKGPVK | VLYVDDSNEN | 120 | |
| GCELDKEIKN | SFNPEAQVFM | PISDPASSVS | SSPSPPFGHS | AAVSPTFMPR | STQPLTFTTA | 180 | |
| TFAATKFGST | KMKNSGRSSK | VARTSPISLG | LNVNVNDLLK | QKAISSSMHS | LYGLGLGSQQ | 240 | |
| QPQPQPQQPP | SQPPPPPPPP | QQQQQHQQQQ | QQQQQQQQQP | QQQTSALSPN | AKEFIFPNMQ | 300 | |
| GQGSSTNGMF | PGDSPLNLSP | LQYSNAFNVF | AAYGGLNEKS | FVDGLNFSLN | NIQYSNQQFQ | 360 | |
| PVMAN | | | | | | 365 | |

Homology comparison shows that human, mouse and rat Tob have very high homology.

1. Homology of cDNA

Rat Tob1 gene cDNA has 90% and 93% homology respectively with human and mouse Tob1 cDNA at the DNA level.

2. Homology of protein

Rat Tob1 protein has 97% and 95% homology respectively with human and mouse Tob1 protein at the amino acid level.

### Practical Example 2

Tissue Distribution of Tob

Northern blotting method: total RNA was extracted from various types of rat tissue, and after denaturing by gel electrophoresis, the RNA was transferred to a nylon membrane, and hybridisation was carried out on the membrane with an α-³²P-dATP labelled probe. The hybridisation results were finally displayed autoradiographically, from which the expression distribution of the target gene in different tissues was discovered.

The results of Northern blotting hybridisation show that the mode of rat Tob1 gene expression is similar to that of humans and mice (FIG. 2).

### Practical Example 3

Distribution of Tob in the Brain

In situ hybridisation:

According to conventional methods, after fixing the animal's brain tissue specimen in paraformaldehyde, it was embedded in paraffin and a section was taken. The tissue section underwent the processes of removal of the paraffin, rehydration, fixing, digesting, post-fixing, and pre-hybridisation. It was hybridised with a Digoxin-labelled probe, after which the distribution of the target gene in the brain was measured through an alkaline phosphatase-linked anti-Digoxin antibody.

The results of in situ hybridisation show that the Tob1 gene has a unique expression in the neurons of the hippocampus in the rat (FIG. 3). This is similar to the Tob gene distribution in the mouse (FIG. 4).

### Practical Example 4

Morris Water Maze Experiment

I. Experimental Equipment and Environment:

Maze diameter=150 cm, height=45 cm;

Platform diameter=10 cm, depth below water level approximately 2 cm;

Video camera, sampling equipment and software,

Water temperature 24-27° C., with the addition of a dye which is harmless to rats; so that the rat is unable to see the platform concealed below the water level.

II. Experimental Animals and Nucleic Acid Sequence:

Male SD rats, bodyweight 180 g-210 g. Divided into three groups, Tob-antisense nucleic acid sequence group 8 animals; random nucleic acid sequence group 8 animals; and blank control group (10 animals).

Tob antisense nucleic acid sequences:

(1) 5′-act tgg att tca agc tgc at-3
(2) 5′-act tct cgt tga ggc ctc cg-3′
5′-gac tga cat gcg att gag ct-3′

Random nucleic acid sequence:

III. Operation:

A catheter was implanted bilaterally into the CA1 region of the hippocampi in rats, used for drug administration in the behavioural experiments. The rats entered the Morris water maze for training 7-12 days after the operation.

IV. Drug Administration:

6 hours before the behavioural training, the drug was administered to the CA1 region. The Tob-antisense sequence and the random sequence concentrations were both 1 nmol/μL, and the dose was 1.5 μL for the CA1 region on each side.

V. Training Method:

For each training session, the rat was placed randomly at four positions in the water maze (as shown in FIG. 5), facing the tank wall (but ensuring that the opportunities for setting out from each of the four locations were equal). It was allowed to search in the water for 60 s, and if it found the platform, it was allowed to stay on the platform for 30 s. After then being returned to its cage to rest for 30 s, the next training session was carried out; if it did not find the platform, it was manually led to the platform, and the next training session began after it had remained there for 60 s. The training was carried out in two steps, and after each rat had been trained for 6 consecutive sessions, it was allowed to rest for 1 hour. The next training step was then carried out, giving a total of 12 training sessions. The time required by the rat to find the platform was recorded.

VI. Test Method:

1. Hidden Platform Test Method:

The test was carried out 48 hours after the training with the objective of measuring the memory retention capability of the rat. The test was carried out for a total of 3 times, with the rat setting out from a fixed location on each occasion. It was likewise allowed to search in the water for 60 s. If it found the platform within 60 s, it would be immediately returned to its cage and after resting for 60 s the next test would begin; if it had not found the platform within the stipulated 60 s, it was led to the platform and allowed to remain there for 30 s, after which it was returned to its cage to rest for 30 s, and then the next test began. The time taken by the rat to find the platform was likewise recorded.

2. Exposed Platform Test Method:

This test was carried out after all the other tests were completed; it was carried out with the platform exposed above the water surface. The objective was to observe whether the rat had any cognitive impediments which would affect its ability to find the platform. The test was carried out for a total of 3 times, and each time the rat set out from a fixed position, but the location of the exposed platform was varied randomly. In each test the rat was first placed on the platform to adapt for 30 s, after which it was placed in the water at the fixed location and allowed to search for 60 s. If it found the platform within 60 s, it would be immediately returned to its cage to rest for 60 s, after which the next test began. If it did not find the platform it would be taken out of the water, placed in its cage to rest for 60 s and then subjected to the next test. The experimental results showed that after the rats in each group had been allowed to adapt for 30 s on the platform, they could all rapidly find the platform, and there was no significant difference between the groups.

VII. Experimental Results:

As shown in FIG. 6. The x axis is the number of training sessions and the memory measurement at 48 hours, and the y axis is the time required by the rats to find the platform (the mean value of the 3 training sessions or tests). After the bilateral injection of the Tob gene antisense nucleic acid sequence into the CA1 region of the hippocampi, there was a significant slowing down in the speed of learning by the rats in the Morris water maze, and the 48 hour memory maintenance ability was also correspondingly lowered.

### Practical Example 5

Electrophysiological Recording of Long-Term Potentiation (LTP) of the Hippocampus CA1 Region

In this practical example, the Tob gene antisense sequence, a random sequence nucleic acid or physiological saline was injected into the CA1 region of the hippocampi of the rat, and the effects were observed on the long-term potentiation (LTP) of the CA1 region synapses. Then the action of the Tob gene on learning and memory was studied from the angle of the plasticity of the synapses in the hippocampi.

I. Experimental Animals and Nucleic Acid Sequences:

Male SD rats, bodyweight 200-250 g. Divided into 3 groups, Tob-antisense nucleic acid sequence group 8 animals; random nucleic acid sequence group 8 animals; and physiological saline control group 6 animals.

Tob antisense nucleic acid sequence:

| | |
|---|---|
| (1) | 5′-act tgg att tca agc tgc at-3′ |
| (2) | 5′-act tct cgt tga ggc ctc cg-3′ |

Random nucleic acid sequence:

5′-gac tga cat gcg att gag ct-3′

II. Operation:

Each rat was anaesthetised with urethane (1250 mg/kg of bodyweight injected intra-abdominally). Then the rat was fixed in a three-dimensional position finder. An incision was made in the scalp, and the surface of the skull was cleaned. Two holes of diameter 1.5 mm were drilled in the P4.9L3.8 and P3.4L2.8 points respectively. The P4.9L3.8 hole was used for insertion of an exciter electrode, and the P3.4L2.8 hole was used for insertion of a recording electrode. The room temperature was maintained at 25 degrees, and the animal's body temperature was maintained at 37 degrees.

III. Exciter and Recorder Electrodes:

The exciter electrode was a concentric cylindrical electrode, with a length of lacquered wire inside a stainless steel needle tube, the internal diameter of the needle tube and the diameter of the lacquered wire being 0.2 mm. The recording electrode was a single pole electrode, made of a single length of lacquered wire of diameter 0.2 mm. The recording electrode and one length of stainless steel tube (external diameter 0.7 mm, internal diameter 0.4 mm) were stuck together. The needle for injection of the drug was passed through this stainless steel tube to reach the recording site.

IV. Recording of Electrical Potential:

1. Positioning of electrodes: the exciter electrode was positioned in the Shaffer channel of the hippocampus, and the recording electrode was positioned in the CA1 region. The Shaffer channel was excited with a single pulse of square wave (wave width 50 μs ²), and the optimum excitatory postsynaptic potential (EPSP) was recorded in the CA1. ² Translator's note: written as “us” in the original, just as the following paragraphs write “ul” where the intended meaning is presumably “microlitre”.

2. Drug administration method: the drug injection needle was passed through the stainless steel tube beside the recorder electrode and inserted into the recording site, and 1.0 μL of antisense sequence nucleic acid with a concentration of 1.0 nmol, or 1.0 μL of random sequence nucleic acid with a concentration of 1.0 nmol, or 1.0 μL of physiological saline was injected at a rate of 0.2 μL/min into the recording site. After 9 hours a further 1.0 μL was injected.

3. Selection of optimum excitation strength: a single pulse square wave (wave width 50 μs) excited the Shaffer channel, and the strength of the excitation was adjusted from small to large (100-800 μA), and the EPSP for the different excitation strengths were recorded. Graphs were drawn of the response strength to the excitation. The optimum excitation strength was taken as ⅔ of the excitation strength to produce the maximum EPSP response.

4. Recording of electrical potential: 2.5 hours after the second injection, the Shaffer channel was excited with a single pulse (50 μs) at the optimum strength, and the EPSP response in the CA1 region was recorded. Excitation and recording were carried out once each minute for 30 consecutive minutes. Then a strong direct excitation of the Shaffer channel was carried out (a series of 20 pulses at intervals of 5 μs with a total of 3 series of excitations with intervals between series of 30 s). After the strong direct excitation, the Shaffer channel was excited as before with a single pulse (50 μs) at the optimum strength, and the EPSP response in the CA1 region was recorded. Excitation and recording were carried out once each minute continuously for 6 hours. The proper time for the strong direct excitation is 12 hours after the first drug administration and 3 hours after the second drug administration. After the 6 hours of recording was complete strong direct excitation of the Shaffer channel was again carried out with the same parameters, and then the Shaffer channel was again excited with a single pulse (50 μs) at the optimum strength, and the EPSP response in the CA1 region was recorded. Excitation and recording were carried out once each minute continuously for 1 hour.

V. Data Processing and Analysis:

Mean overlaying was carried out on the EPSP waveforms for 5 or 10 successive recordings, and after calculating the mean overlay, the gradient of the rising phase of the EPSP potential was calculated. With the mean value of the EPSP gradient before the strong direct excitation as 100%, the EPSP gradient at each time point before and after the strong direct excitation was calculated. A graph was drawn of the EPSP gradient against time.

VI. Experimental Results

The experimental results are shown in FIG. 7. After injection of the Tob gene antisense nucleic acid, the long-term potentiation (LTP) of the synapse potentials in the CA1 region were significantly less than the random sequence control group or the physiological saline control group LTPs, indicating that the CA1 Tob gene participated in the induction and maintenance of the long-term potentiation.

### Practical Example 6

Effect on Mouse Memory of Measured Tob Gene in the Step Down Test

For step down behaviour the step down method (Step Down Test) can be used [Chen Shuangshuang, Guan Linchu, Bao Shimin, Jin Meilei, Comparative Study on Open Space Behaviour and Memory in Four Breeds of Mice, Psychological Science 1994 Vol. 17 No. 1, pp. 39-41] to conduct the test: the experimental apparatus was a 20×28×30 cm transparent plastic box, the base of which was a metallic circuit board, with a 8×8×1.5 cm platform placed in the left corner as a safe area. Before the experiment began, training was first carried out: a mouse was placed in the experimental box and allowed to move around freely for 1 minute, after which the mouse was gently chased onto the small platform (the mouse would generally come down off the platform rapidly), and the number of seconds was recorded between when the mouse had fully climbed onto the small platform to when it came down (latent period). This was repeated three times in succession, after which the mouse was again chased onto the small platform, and current was immediately passed through the circuit board on the base of the box (50 V, 0.5 mA). After the mouse had again jumped off the small platform and received an electric shock, it would jump back onto the platform to avoid the shock, at which point it was regarded as having been trained. The mouse was taken out and placed in a feeding cage. 24 hours after training, the mouse was again placed on the small platform in the experimental box, and current was passed through the circuit board in the base of the box. The time in seconds between the mouse being placed on the small platform to coming down was recorded. 48 hours after training a single interval was recorded in the same way. In order better to observe and evaluate the animals' memory behaviour, the number of trainings and the observations after 48 hours were increased in accordance with the relevant literature.

In this practical example, BALB/c mice+saline solution were used for Group 1; BALB/c mice+randomly sequenced nucleic acid were used for Group 2; and different breeds of mice+Tob antisense sequence nucleic acid were used for Groups 3-9. The Tob antisense nucleic acid sequence and the random nucleic acid sequence were the same as in Practical Example 4.

The results are shown in FIG. 8A (24 hours) and 8B (48 hours). The antisense nucleic acid experimental groups showed a significant reduction in memory in comparison with the control groups, and it could significantly inhibit mouse leaning and memory in all the breeds.

1. Method for diagnosing an individual's susceptibility to amnesia, characterised by including the steps: detecting that individual's Tob gene, transcript and/or protein and comparing with normal Tob gene, transcript and/or protein, Differences present indicating that the probability of that individual suffering from amnesia is higher than that of the normal population.

2. Method according to claim 1, characterised by the detection of the human Tob gene or transcript and its relative difference from a normal human Tob nucleotide sequence.

3. Method for treating amnesia, characterised by including the step of using a safe and effective dose of normal Tob protein on the patient requiring the said treatment.

4. Method for improving the memory or movement coordination of a mammal characterised by including the step of using a safe and effective dose of Tob protein on the subject requiring it.

5. Tonics characterised by including a mammalian Tob protein or its active fragment.

6. Pharmaceutical compositions characterised by including a safe and effective dose of a mammalian Tob protein and a pharmaceutically acceptable carrier.

7. Said pharmaceutical compositions according to claim 6, characterised by the said Tob protein being selected from the following groups of animal Tob protein: human, rat and mouse.

8. A reagent kit for detecting susceptibility to amnesia, characterised by including an initiator for the specific amplification of the Tob gene or transcript, or an antibody specifically binding with the Tob protein.

9. Method for screening drugs for the treatment of amnesia, characterised by including the steps:
(1) Inserting the Tob gene cDNA into an expression vector, transfecting a mammalian cell strain and preparing a cell strain to express the Tob protein;
(2) Adding a test compound to the culture fluid for the cell strain expressing the Tob protein in step (1) to detect changes in the amount of Tob protein expressed; the compound promoting the increase in the amount of Tob protein expression being the drug awaiting screening for the treatment of amnesia.

10. A type of isolated Tob protein characterised by including the amino acid sequence indicated by SEQ ID NO: 2.
